(19) United States
(12) Patent Application Publication
(10) Pub. No.: US 2011/0212881 A1
(43) Pub. Date: Sep. 1, 2011

(54) ALEURONE AS A PREBIOTIC FIBER FOR IMPROVED INTESTINAL HEALTH

(75) Inventor: William Ronald Aimutis, Blaine, MN (US)

(73) Assignee: Cargill Incorporated, Wayzata, MN (US)

(21) Appl. No.: 13/128,138

(22) PCT Filed: Nov. 17, 2009
(86) PCT No.: PCT/US09/64787

§ 371 Date: May 6, 2011

Related U.S. Application Data
(60) Provisional application No. 61/200,099, filed on Nov. 24, 2008.

(51) Int. Cl. A61K 38/00; A61P 1/00; C07K 14/415

(52) U.S. Cl. 514/1.1; 530/375

(57) ABSTRACT

The present invention is directed to methods of treating, maintaining or improving the feces quality and gastrointestinal tract health of mammals. The present invention is further directed to food products that can be used to treat, maintain or improve the feces quality and gastrointestinal tract health of mammals. In at least one aspect of the invention, the method includes introducing aleurone into the diet of a mammal. In another aspect the food product includes aleurone.

### CROSS REFERENCE TO RELATED APPLICATION

This application claims the benefit of the U.S. Provisional Patent Application Ser. No. 61/200,099, filed 24 Nov. 2008, which is hereby incorporated by reference in its entirety.

### FIELD OF THE INVENTION

The invention relates to a food product containing wheat aleurone. The invention further relates to a food product containing wheat aleurone in an amount that provides good feces quality, good gastrointestinal health or a combination of both feces quality and gastrointestinal health of humans and animals. The invention also relates to methods of maintaining and/or improving feces quality, gastrointestinal tract health or a combination of both feces quality and gastrointestinal tract health of humans and animals. The invention additionally relates to a method of treating a mammal by introducing aleurone into the diet of the mammal.

### BACKGROUND OF THE INVENTION

Whole grains are rich sources of fermentable carbohydrates including dietary fiber, resistant starch and oligosaccharides. Dietary fiber, as used herein, is the carbohydrate and carbohydrate digestion products that are not absorbed in the small intestine of healthy humans but enter the large bowel. This includes resistant starches, beta-glucans and other soluble and insoluble carbohydrate polymers. It is thought to comprise that portion of carbohydrates that are fermentable, at least partially, in the large bowel by the resident microflora. Undigested carbohydrate that reaches the colon is fermented by intestinal microflora to short chain fatty acids and gases. Short chain fatty acids (SCFAs) include acetate, butyrate, and propionate, with butyrate being a preferred fuel for the colonic mucosa cells. Short chain fatty acid production has been related to lowered serum cholesterol and decreased risk of cancer. Undigested carbohydrates increase fecal wet and dry weight and speed intestinal transit.

In wheat, the majority of desirable “whole grain” components are concentrated in the aleurone layer, the primary component of bran. Aleurone is an extremely nutritional grain component, in particular of wheat grain. Aleurone is present in wheat grain as a single-cell layer (aleurone layer) between the flour body (endosperm) and shell (pericarp and testa). The percent by weight of aleurone in wheat grain averages at about 8%.

Aleurone is isolated from bran and further processed using physical, such as mechanical-abrasive and biological-enzymatic, methods. Bran used as the parent material for this purpose is obtained in a conventional manner in a grain mill.

Aleurone cells of wheat grain contain the most important nutritional substances in concentrated form, such as vitamins, minerals, sterols, essential fatty acids, nutrient fibers, high-quality protein (albumin), along with special protective substances (bioactive substances, such as polyphenols, lignan, phytin, etc.).

Aleurone contains approximately 47% fiber, with the breakdown being about 43% insoluble fiber and about 4% soluble fiber. The nutrient fibers in aleurone may improve digestion in the large intestine (prebiotic). In addition, ion exchange activity slows resorption in the small intestine, and binds undesired substances. In so doing, they contribute to a long-lasting feeling of satisfaction and detoxification.

Aleurone bioactive substances, such as polyphenols, flavonoids, lignan, beta-glucan, etc., help protect against several illnesses afflicting human civilization, such as arterial disease and certain forms of cancer.

It has been surprisingly discovered that products made with aleurone, such as wheat aleurone, provide one or more health benefits when incorporated into the diets or otherwise delivered to the gastrointestinal tract of mammals such as humans and animals. Examples of these health benefits include bowel health and metabolic health.

### SUMMARY OF THE INVENTION

This invention provides methods of treating, maintaining or improving the feces quality and gastrointestinal health of mammals including companion pets, and also provides related food products. In one aspect the invention includes treating a mammal by introducing wheat-derived aleurone into the diet of the mammal. The aleurone may be introduced in a manner and amount effective to improve good feces quality and/or to improve gastrointestinal tract health. The aleurone can be effective when ingested orally by mammals, including dogs, cats and horses.

In one aspect the aleurone is introduced in an amount of about at least 0.1 to 2% of the mammal's total daily food intake, or in an amount of at least 0.1% to 2% of the mammal's total daily food intake. In another aspect the aleurone is introduced in an amount of about 0.5 to 1.5% of the mammal's total daily intake, or in an amount of 0.5% to 1.5% of the mammal's total daily food intake; or in an amount of about 1% of total intake, or in an amount of 1% of total intake. In another aspect an effective amount of aleurone is delivered to the gastrointestinal tract of a companion animal as a method of improving one or more indicators of bowel health or metabolic health. Such indicators can include, for example, decreased pH of the bowel contents, increased total SCFA concentration or total amount of one or more SCFAs in the bowel contents, increased fecal quality, increase in total water volume of bowel or feces, improved laxation, increase in number of activity of one or more species of probiotic bacteria, increase in fecal bile acid excretion, reduced urinary levels of putrefactive products, reduced fecal levels of putrefactive products, increased proliferation of normal colonocytes or any combination of these indicators. Increased fecal quality can include a smooth, shiny appearance.

In another aspect the invention includes a food product or composition having wheat-derived aleurone in an amount that provides good feces quality, good gastrointestinal health or a combination of the same. And another aspect includes a pet food composition having wheat-derived aleurone. In yet another aspect the invention may be applied in connection with other animals such as poultry and fish. In an additional aspect the invention includes a food product suitable for humans and animals including wheat-derived aleurone in the food product in an amount that provides good feces quality, good gastrointestinal health or a combination thereof. In an aspect the food product is suitable for animals such as companion animals, poultry and fish.

### DETAILED DESCRIPTION OF THE DRAWINGS

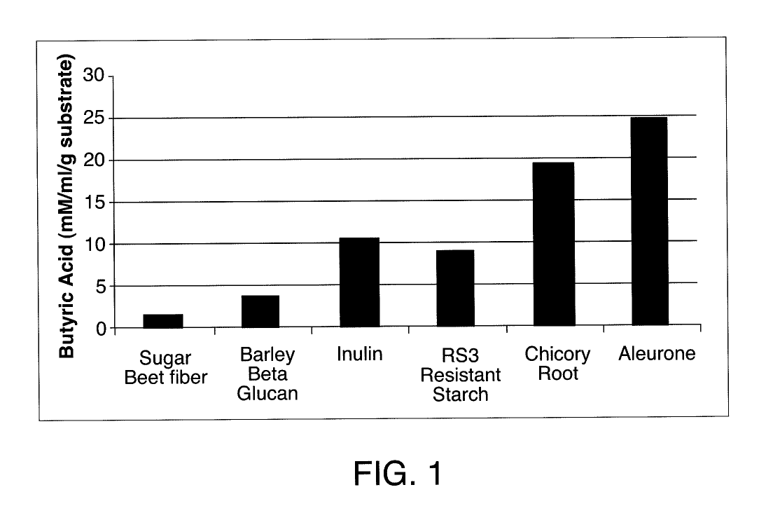

FIG. 1 illustrates butyric acid production by fibers screened in this study.

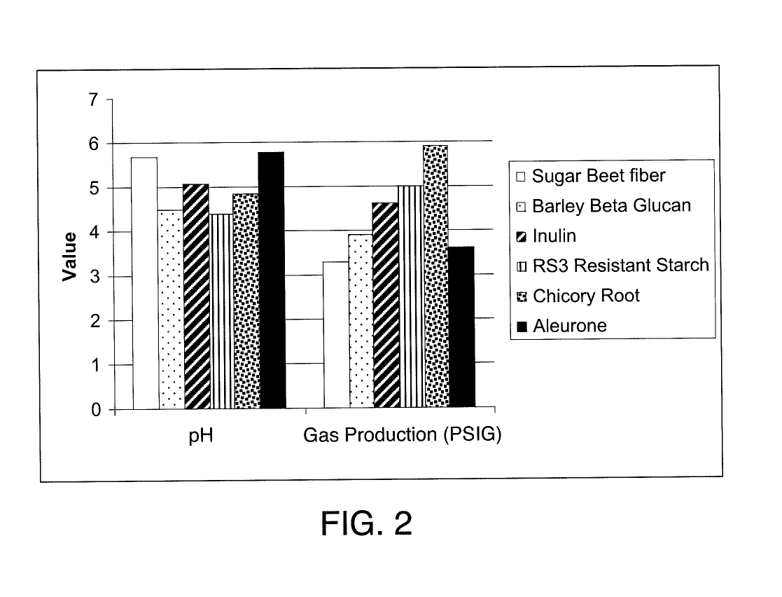

FIG. 2 illustrates the observed pH values and gas pressure produced after fermentation by canine fecal flora of screened fiber sources.

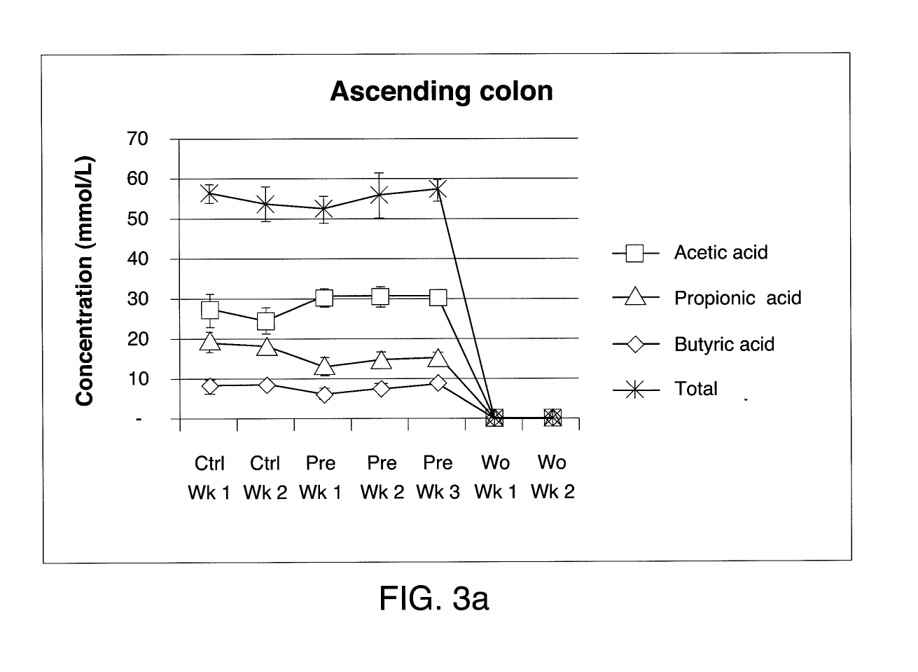

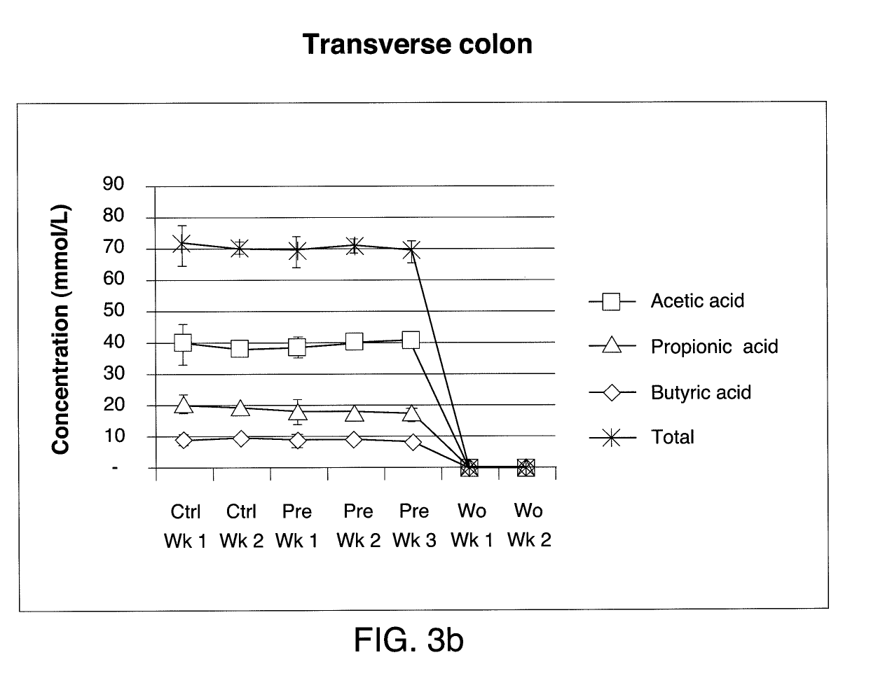

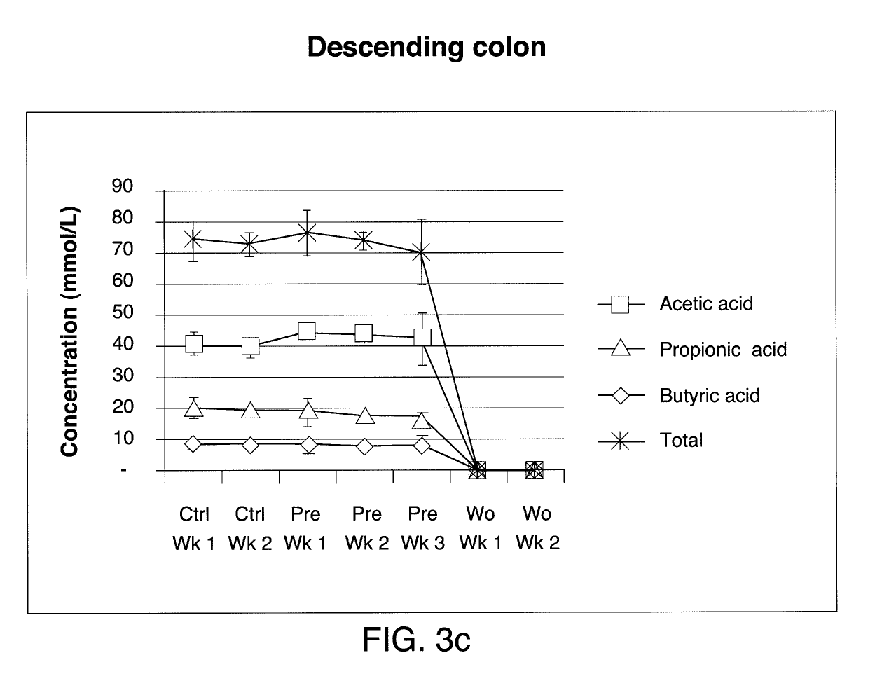

FIGS. 3a, 3b, 3c illustrates the volatile fatty acids produced during fermentation of aleurone by human fecal flora after fermentation in a dynamic in vitro gastrointestinal system in, respectively, the ascending colon, the transverse colon and the descending colon.

### DETAILED DESCRIPTION OF THE INVENTION

In one aspect, the invention is a food product containing wheat aleurone. In another aspect, the invention is a food product containing wheat aleurone in an amount that provides good feces quality, good gastrointestinal health or a combination of both feces quality and gastrointestinal health of mammals, such as humans and animals. In another aspect, the invention is a method of maintaining and/or improving feces quality, gastrointestinal tract health or a combination of both feces quality and gastrointestinal tract health of mammals, such as humans and animals.

In one aspect of the invention, aleurone is incorporated into the diet or otherwise introduced to the gastrointestinal tract of a mammal to maintain and/or improve the feces quality and/or gastrointestinal tract health of an animal as measured by several indicators. In another aspect of the invention, aleurone is incorporated into the diet of companion animals, such as canines, as results in the improvement of bowel health as measured by several indicators. The indicators may include, but are not limited to:

i) decreased pH of bowel contents,
ii) increased total SCFA concentration or total SCFA amount in the bowel contents,
iii) increased concentration or amount of one or more SCFAs in the bowel contents,
iv) increased fecal bulk,
v) increase in total water volume of bowel or feces, without diarrhea,
vi) improved laxation,
vii) increase in number or activity of one or more species of probiotic bacteria,
viii) increase in fecal bile acid excretion,
ix) reduced urinary levels of putrefactive products,
x) reduced fecal levels of putrefactive products,
xi) increased proliferation of normal colonocytes,
xii) reduced inflammation in the bowel of individuals with inflamed bowel,
xiii) reduced fecal or large bowel levels of any one of urea, creatinine and phosphate in uremic patients, or
xiv) any combination of the above.

In one aspect of the invention, the pH of bowel contents may be reduced by at least 0.1 pH units. In a further aspect of the invention, the pH of bowel contents may be reduced by at least 0.2 pH units.

The Short Chain Fatty Acids (SCFA), the concentration or total amount of which may vary, can be any one or more of formate, acetate, propionate, butyrate, succinate or branched forms thereof. In one aspect of the invention, the SCFA is one or more of acetate, propionate or butyrate. In another aspect of the invention, the SCFA is butyrate. In still another aspect of the invention, the SCFA is selected from formate, acetate, propionate, butyrate, succinate or branched forms thereof. In yet another aspect of the invention, the SCFA is acetate, propionate, butyrate or combinations thereof. In another aspect of the present invention, the SCFA is butyrate. Alternatively, the change in concentration or total amount is a pooled value of total SCFA or a selected group of one or more of them. The concentration change may be as measured in feces, or internally, which may be in the caecum, the proximal colon, the distal colon or any combination of these. The total amount may increase while the concentration remains the same or even increases if the bowel contents increase in volume over time.

Among probiotic bacteria, bifidobacteria species may be the most prominent, however, lactic acid bacteria are similarly included such as, for example, Lactobacillus bulgaricus, Lactobacillus acidophilus, Lactobacillus johnsonii, Lactobacillus casei, Lactobacillus plantarum; or Enterococcus faecium or Streptococcus thermophilus.

Fecal bulk increases principally as a result of greater numbers of bacteria that are supported in the caecum and colon. The volumes may be measured by an increase in quantity of feces, or may be measured in situ by estimating the volume of cecal, proximal colon, or distal colon contents, separately or as a combination of two of these or all three of these.

The water volume of the bowel or feces generally increases as a result of increased number of bacteria. The water content may be measured by comparing the wet weight of the feces or bowel contents with dry weight after drying, the volume of water can be calculated from this decrease in weight.

Laxation relates to the passage of solids from the bowel, and entails measuring defecation in a quantitative and/or qualitative manner. Frequency of defecation is one aspect of laxation. One qualitative measure relates to hardness of stools, whereby passage is easier, in contrast to constipation, but where stools are not so soft or loose as to constitute diarrhea.

Probiotic bacteria are generally recognized as good for bowel health, being non-infectious and producing beneficial metabolites from their fermentation activities. Among the probiotic bacteria, bifidobacterial species are the most prominent. Lactic acid bacteria are similarly included; for example, Lactobacillus bulgaricus, Lactobacillus acidophilus, Lactobacillus casei, Lactobacillus johnsonii, Lactobacillus plantarum, Enterococcus faecium or Streptococcus thermophilus. Numbers of individual species or genera might individually or collective increase. There may also be a reduction in the number of bacterial species that have a potentially adverse effect on the large bowel. Many such species are unable or less able to utilize aleurone for energy compared to the probiotic organisms. Examples of such adverse bacteria include some Clostridium, Veillonella and Klebsiella.

Aleurone may impact fecal bile acid excretion. Increased fecal bile acid excretion induces the liver to produce more bile acids, utilizing cholesterol as a substrate in the production of the bile acids. The liver can obtain cholesterol for the synthesis of bile acids from the blood, lowering blood cholesterol concentrations. Alternatively this may be used as a general marker of bowel activity, and clearance of bile. The build up of bile acids is also thought to have at least a correlation to bowel pathogenesis.

Aleurone may also reduce urinary and fecal levels of putrefactive products or indicators of putrefactive products. This is indicative of a reduced level of fermentation by putrefying bacteria in the colon or caecum. Additionally these may be indicative of reduced small intestinal overgrowth. The level of these compounds can be measured for, by example using GC-MS or other techniques. Many of these compounds are metabolic products or byproducts of protein or amino acid degradation. Compounds may be urea, ammonia and other waste nitrogen products or sulfides and sulfur containing compounds including hydrogen sulfide gas. Specific compounds that may be tested include but are not limited to phenol, indole, skatole, and ammonia, p-cresol, 4-ethylphenol, urea, ketones, and amines.

In another aspect of the present invention, aleurone acts as a prebiotic to influence growth of indigenous lactic acid bacteria and other beneficial fermentative bacterial species. The benefits to mammals such as humans and companion animals include improved nutrient digestibility. In at least one aspect, the improved nutrient digestibility improves nutritional well being in the gastrointestinal tract including optimal fecal mass, less flatulence, and lower malodorous compounds in the feces.

A complex community of microorganisms colonizes the mammalian gastrointestinal tract from the mouth to anus, but the colon is the main site of this microbial colonization and metabolism. Ileum passage of aleurone initiates digestion and metabolism by the anaerobic microflora of the caecum and colon that produce enzymes necessary for polysaccharide hydrolysis and catabolism. Breakdown is affected by bacterial species very similar to those found in the rumen of obligate herbivores and with very similar products: gases, such as carbon dioxide, methane and hydrogen, and short chain fatty acids (SCFA). The principle SCFAs formed are acetate, propionate and butyrate in the rough molar proportions 60:20:20. These three acids contribute about 80-90% of total colonic SCFA, the remainder being branched chain and other fatty acids formed from breakdown of dietary and endogenous protein. Animals fed a variety of fibers have shown higher colonic SCFA and in some cases increased bacterial mass in the colon. Many of the effects of aleurone in the colon are probably mediated through SCFA.

Fecal bulk is directly related to increases in microbial mass from undigestible carbohydrate fermentation, which is a large part of the stool weight. Bacteria are about 80% water and resist dehydration; as such they contribute to water holding in fecal material. The number of bacteria in human feces is approximately 4−8×10¹¹/g dry feces, and makes up about 50% of fecal solids in human subjects on a Western diet. Gas production from colonic fermentation can also have some influence on stool bulk by trapping gas in the stool bulk to increase volume and decrease fecal transit time.

Metabolic fermentation end products, namely the gases, SCFA and increased microflora, play a role in the physiological effects of indigestible carbohydrate in the colon and have implications for local effects in the colon and systemic effects. The gases produced from fermentation by strict anaerobic species such as Bacteroides, some non-pathogenic species of clostridia and yeasts, anaerobic cocci and some species of lactobacilli are mostly released as flatulence or are absorbed and subsequently lost from the body through the lungs. However, some of the hydrogen and carbon dioxide produced from these microflora may be further metabolized to methane (CH₄) by methanogenic bacteria, thus reducing intestinal gas pressure. Of these anaerobic microorganisms, the clostridia, eubacteria and anaerobic cocci are the most gas producing, while the bifidobacteria do not produce any gases.

Prebiotics are selectively fermented ingredients that allow specific changes both in the composition and/or activity of the gastrointestinal microflora that confers benefits. Prebiotic activity has historically been characterize by influencing growth and metabolism of beneficial bacteria, such as bifidobacteria and lactobacilli. Multiplying beneficial bacteria reduce colonic pH, making the environment less inviting for potentially harmful bacteria such as E. coli, clostridia, Veillonella and Klebsiella. Proliferation of beneficial bacteria provides significant health effects, including enhanced digestion and improved lactose tolerance, promoting recycling of compounds such as estrogen, synthesizing vitamins, especially B-group vitamins, producing immune-stimulating compounds, inhibiting growth of harmful bacteria, reducing production of toxins and carcinogens, restoring normal intestinal bacteria during antibiotic therapy, and reducing the potential for several pathologies commonly associated with higher numbers of pathogenic intestinal bacteria.

The primary SCFA generated by fermentation are acetate, propionate and butyrate, accounting for about 83-95% of the total SCFA concentration in the large intestine, which ranges from about 60-150 μmol/L. In most mammalian species concentrations of these acids are highest where microflora concentrations are also highest, namely in the cecum and right or transverse colon. Corresponding to these higher acid levels, the pH is also typically lowest in the transverse colon (5.4-5.9) and gradually increases through the distal colon to 6.6-6.9. As the pH is reduced, the colonic environment becomes less favorable for toxin producing and ill health promoting microflora, such as E. coli, clostridia, and certain yeasts.

Dietary fiber has been reported for prevention and management of simple constipation. Fibers vary in their effects on bowel function. Cereal brans such as wheat and rice brans are high in insoluble non-starch polysaccharides and appear to be more effective at improving laxation by shortening transit time, softening stools through raised water holding, increasing stool volume and weight in the form of bacteria and undigested and non-fermentable material.

The role of fiber in maintenance of colonic mucosal integrity is understood imperfectly. Experiments with animal models such as pigs have shown that the weight and thickness of the colon is increased with diets high in fiber—consistent with greater cell growth. Butyrate is thought to play a more critical role in the cell biology of colonocytes and is preferred over acetate and propionate as their oxidative fuel. Butyrate inhibits proliferation of malignant cells from the human colon in vitro via inhibition of DNA synthesis. In addition, induction of cell differentiation has also been reported, an observation consistent with the fact that when cells differentiate they lose their capacity to proliferate. Butyrate also enhances the capacity of colonic cells to repair DNA damage. All of these effects require physical presence of the acid and are obtained at butyrate concentrations similar to those found in the colon in vivo. It is reported butyrate enhances proliferation of normal cells but may exert antineoplastic effects on susceptible cells and significantly retards growth of human colon cancer cells in vitro.

In one aspect, the invention identifies fiber sources capable of improving one or more indicators of bowel health or metabolic health in a mammalian animal by studying their effect in vitro on canine fecal flora fermentation. Various aspects were studied in vitro including post fermentation gas pressure, pH, volatile fatty acid concentration, and malodorous compound concentration. The field was narrowed and using fecal flora from several different canine breeds confirmed aleurone was a fiber capable of influencing bowel health. In another aspect, aleurone is shown to support in vitro growth of several probiotic species in pure culture. In still another aspect, aleurone is shown to support in vitro growth of bifidobacteria species in a mixed human fecal fermentation. In yet another aspect, aleurone is incorporated into canine feed. In this aspect aleurone is well tolerated and improves fecal characteristics including growth promotion of indigenous and probiotic bacterial species and improved stool moisture content.

The method of feeding companion animals may comprise the step of administering aleurone to the animal, in one or more doses, in an amount and for a period of time whereby the level of the one or more of the bowel health or metabolic indicators improves. The indicator may change relative to consumption within a time period of hours, as in the case of some of the indicators such as pH, elevation of levels of SCFA, post-prandial glucose fluctuation, or it may take days such as in the case of increase in fecal bulk or improved laxation, or perhaps longer in the order of weeks or months such as in the case where the butyrate enhanced proliferation of normal colonocytes is measured. The preferred method of administering aleurone to companion animals is through their daily feeding regimen for as long as they consume the product containing aleurone.

Dosages may vary depending on the size of the animal ingesting aleurone. Typically for dogs the target is 0.15 grams/kg body weight/day. The aleurone of the present invention can be readily incorporated into food products at levels typically ingested in normal companion animal diets. Intake of at least about 1.75 g per day is thought to provide a measurable benefit, although more preferably the intakes are at least about 20-30 grams of the altered wheat starch per day. Typically, humans have daily intakes of at least 100 to 200 g of starchy food products such as bread or pasta, which means that levels of altered starch in the food product of at least 5 to 10% will typically provide a beneficial effect. It is proposed that levels of less than that, for example, as low as 1% will also give a beneficial effect which may or may not be immediately measurable.

While the previous description is directed to the feeding of canines, it is to be understood the invention is also applicable to other companion animals, livestock, as well as humans. Examples of livestock animals include but are not limited to cows, sheep, pigs, horses, and examples of companion animals include but are not limited to dogs and cats. The method may be particularly applicable to non-ruminant mammals or animals such as mono-gastric mammals. The invention may also be applicable to other agricultural animals for example poultry including, for example, chicken, geese, ducks, turkeys, or quails, or fish.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

As used herein, a recitation of a range of values is merely intended to serve as a shorthand method of referring individually to each separate value falling within the range, and each separate value is incorporated into the specification as if it were individually recited herein.

### EXAMPLES

The following examples are representative of the invention, and are not intended to be limiting to the scope of the invention.

### Example 1

### Screening of Fibers

### In Vitro

Dietary fibers are formulated into companion animal diets to improve gut microbial ecology, improve stool quality, increase mineral absorption, and improve immune status. Many soluble fibers are not hydrolyzed in the mammalian small intestine but are fermented rapidly in the lower gastrointestinal tract. Oligosaccharides from these fibers are fermented to yield short chain fatty acids (SCFA). Research has indicated SCFA may have a profound effect on gut morphology and cellular turnover. One desirable fatty acid is butyric acid. Butyrate serves as a nutritional source for coloncytes (Sakata, 1987), and has been shown to be inhibitory to adverse cell proliferation and cyclo-oxygenase activity. Butyrate inhibits apoptosis of colon crypt cells in vivo so that less tryptophan from cell debris is available for skatole formation in the pig colon (Claus et al., 2003). Skatole is implicated in colon cancer and foul smelling feces.

Substrates and Donors. The following substrates were screened in this study: sugar beet fiber (Cargill), aleurone (Cargill), chicory, inulin (Cargill), RS3 resistant starch (Cargill C*Actistar RM 11700), and barley beta glucan (Cargill). Several of these fibers sources were ground using appropriate methods to a suitable particle size. Healthy adult dogs served as sources of fecal material. All dogs consumed commercial dog foods daily.

Fermentation. Fibers (200 mg) were anaerobically fermented in sealed septum bottles at 39° C. with 1% inoculum of fresh fecal microflora obtained from donor animals. Freshly voided dog feces were diluted (1:10) in warmed (39° C.) physiological saline to use as inoculum. The fermentation buffer composition is in Table 1 below. A control blank (containing no fibers) was included. Samples (1 ml) were aseptically withdrawn at 0 and 48 hr for analysis. Prior to withdrawing samples at 48 hr, a digital pressure gauge (Omega Engineering, Model DPG1001B-100G) fitted with a hypodermic needle measured gas production resulting from the fecal fermentation.

TABLE 1
Fermentation Buffer Composition

| Ingredient | Amount, unit/L | |
|---|---|---|
| Na₂CO₃ | 4.0 | g |
| Cysteine HCl | 600 | mg |
| Trypticase | 500 | mg |
| Yeast Extract | 500 | mg |
| (NH₄)₂SO₄ | 480 | mg |
| NaCl | 480 | mg |
| K₂HPO₄ | 292 | mg |
| KH₂PO₄ | 292 | mg |
| Trace Salt Solution ¹ | 1 | ml |

¹ Trace salt solution: MgSO₄, 1%; MnCl₂, 2%; FeCl₃, 0.135%; and, CaCl₂, 0.04%.

Chemical Analyses. Aliquots (1 ml) were centrifuged at 13,000×g at 22° C. for 3 min. Supernatants were acidified with an equal volume of phosphoric acid (10 N), and frozen until analysis. Gas chromatography was used to quantify acetate, propionate, butyrate, and lactate concentrations in cell-free, acidified supernatants.

### Results and Discussion

Aleurone fermented by canine fecal flora in vitro had greater butyric acid in the fermentates than other known prebiotic substrates such as chicory root, inulin, and resistant starch (FIG. 1). In addition, gas production was lower when aleurone was utilized as a sole carbon source compared to other prebiotic fibers (FIG. 2). This should result in less flatulence in dogs consuming aleurone as a fiber source. Additionally, pH was not drastically affected by aleurone during fermentation (FIG. 2). The reason for a higher pH after aleurone fermentation is that aleurone is only approximately 50% carbohydrate compared to the other fibers at much higher sugar levels.

These results show aleurone is effective at potentially increasing butyric acid in the gastrointestinal tract of dogs consuming this fiber.

### Example 2

### Ability of Aleurone to Promote Growth of Probiotic Bacteria In Vitro in a Pure Culture

Our objective in this example was to demonstrate aleurone is a prebiotic for various known probiotic strains (Huebner, J, R L Wehling and R W Hutkins (2007) Functional activity of commercial prebiotics. Intl. Dairy J. 17:770-775).

### Materials and Methods

The ability of aleurone to promote growth of known probiotic strains was tested by inoculating a basal MRS medium with no carbohydrate, glucose (0.65%), inulin (0.65%), or aleurone (1%) as the sole carbon sources and incubating for 24 hours at 37° C. The probiotic strains tested were Enterococcus faecium SF-68 (Medipharm), Bifidobacterium animalis ssp. lactis Bf-6 (Cargill), Lactobacillus johnsonii La-1 (Cargill), Lactobacillus casei LCV-1 (Cargill), and Lactobacillus rhamnosus LBCR-1 (Cargill). Prebiotic activity was determined as the ratio of growth on aleurone vs. growth on glucose according to the following formula:

[(log cfu/ml aleurone)₂₄ₕ−(log cfu/ml aleurone)₀ₕ]/[(log cfu/ml glucose)₂₄ₕ−(log cfu/ml glucose)₀ₕ]. Inulin's prebiotic index was calculated in a similar manner by using cell counts for strains grown on inulin as opposed to aleurone.

### Results and Conclusions

In this experiment we evaluated in vitro the prebiotics aleurone (Grainwise® wheat aleurone) and inulin, in comparison to the simple carbohydrate glucose for their ability to stimulate growth of single probiotic strains. The fibers were compared to glucose on an equivalent glucose basis.

All probiotic strains tested utilized the prebiotics aleurone and inulin (Table 2), as measured by utilizing their cell counts after 24 hours of fermentation in MRS broth containing the individual carbon sources to calculate the prebiotic activity score. Growth on aleurone and inulin was less than on glucose for all probiotic strains. Prebiotic indexes indicated aleurone is a suitable prebiotic to support growth of probiotic organisms in vitro. Inulin has been previously reported to have positive prebiotic effects both in vitro and in vivo. However, in these experiments, inulin is suitable as a prebiotic for strains SF-68 and LCV-1, marginal for strains La-1 and LBCR-1, but is minimally a prebiotic for strain Bf-6.

TABLE 2
Prebiotic activity score of probiotic strains incubated
in MRS-glucose, MRS-aleurone, or MRS-inulin broths.

| | Prebiotic | Prebiotic | Prebiotic |
|---|---|---|---|
| | Activity | Activity | Activity |
| | (aleurone | (inulin | (aleurone |
| | compared to | compared to | compared to |
| Strain | glucose) | glucose) | inulin) |
| Enterococcus | 0.904 | 0.856 | 1.043 |
| faecium SF-68 | | | |
| Bifidobacterium | 0.810 | 0.438 | 1.864 |
| animalis ssp. lactis | | | |
| Bf-6 | | | |
| Lactobacillus | 0.876 | 0.622 | 1.452 |
| johnsonii (La-1) | | | |
| Lactobacillus casei | 0.801 | 0.801 | 0.993 |
| LCV-1 | | | |
| Lactobacillus | 0.794 | 0.727 | 0.945 |
| rhamnosus LBCR-1 | | | |

### Example 3

### Ability of Aleurone to Promote Growth of Lactic Acid Bacteria In Vitro with a Mixed Human Fecal Flora Inoculation

The objective of this example was to determine in vitro if aleurone has prebiotic activities in the adult human gastrointestinal tract.

### Materials and Methods

Possible prebiotic properties for the adult human gastrointestinal tract were evaluated by a 3-week administration of aleurone to the Simulator of the Human Intestinal Microbial Ecosystem (SHIME). The dosage rate was 2.5 g/d for each compound.

The reactor setup was adapted from the SHIME as described by Molly et al. (Molly et al. 1993). The SHIME consists of a succession of five reactors simulating the different parts of the human gastrointestinal tract. The first two reactors are of the fill and draw principle to simulate different steps in food uptake and digestion, with peristaltic pumps adding a defined amount of SHIME feed (140 mL 3×/day) and pancreatic and bile liquid (60 mL 3×/day), respectively to the stomach (V1) and duodenum (V2) compartment and emptying the respective reactors after specified intervals. The last three compartments are continuously stirred reactors with constant volume and pH control. Retention time and pH of the different vessels are chosen in order to resemble in vivo conditions in the different parts of the gastrointestinal tract. Overall residence time of the last three vessels, simulating the large intestine, is 76 h. Upon inoculation with fecal microbiota, these reactors simulate the ascending (V3), transverse (V4) and descending (V5) colon. Inoculum preparation, retention time, pH, temperature settings and reactor feed composition were previously described by Possemiers et al. (2004).

The experiment consists of four stages:

Stabilization period: For the evaluation of prebiotics, the colon compartments of the SHIME reactors was first inoculated with an isolated fecal microbial community of a selected healthy volunteer. The SHIME reactor was operated under nominal conditions to stabilize the microbial community and let it adapt its metabolic activity and community composition to the conditions prevailing in the respective colon compartments. This stabilization period lasted for 3 weeks.

Basal period: During the basal period, the SHIME reactor was operated under nominal conditions. Parameters such as short chain fatty acid (SCFA) production and ammonium production was determined 3 times/week and plate count analysis was performed once a week for selected bacterial groups. The results of these analyses served as the background values to be used to compare the measured parameters from the treatment period. The basal period lasted 2 weeks.

Treatment period: During the treatment period, the SHIME reactor was operated under nominal conditions, but with a modified diet containing a lower amount of starch in the medium compared to that of the basal period. In parallel, the diet of the SHIME was supplemented with aleurone. The dosage rate was set at 2.5 g/d. SCFA and ammonium production was determined 3 times/week and plate count analysis was performed once a week. This treatment period lasted for 3 weeks.

Washout period: During the washout period, the SHIME reactor was operated under nominal conditions, with the initial diet. SCFA and ammonium production were determined 3 times/week and plate count analysis was performed once a week. Analysis of these microbial parameters allowed assessing whether changes from the treatment period normalize to the levels of the basal period.

Analysis of the Microbial Community Composition and Activity

During the control, probiotics period, and washout microbial composition and activity were monitored at specific time points to investigate the effect of the probiotic treatment on these microbial parameters.

### Microbial Community Composition

Plate counts: samples were taken 1×/week from all colon compartments.

Investigated groups were: total aerobes, total anaerobes, bifidobacteria, and lactobacilli.

### Microbial Community Activity

Short chain fatty acids (SCFA): samples were taken 3×/week from all colon compartments.

### Results and Discussion

Table 3 describes the effect of aleurone on counts of 4 bacterial groups from different SHIME compartments representing the ascending, transverse, and descending colon. None of the counts were significantly different between the control and treatment period, but an increase in bifidobacteria was observed with this mixed flora population.

TABLE 3
Selected bacterial counts from the SHIME Colon Compartments.

| | | Ascending | Transverse | Descending |
|---|---|---|---|---|
| | | Colon | Colon | Colon |
| | | (Log 10 | (Log 10 | Log 10 |
| | | CFU/ml) | CFU/ml) | CFU/ml) |
| Total aerobes | Control | 6.87 | 6.95 | 6.78 |
| | Aleurone | 6.58 | 6.69 | 6.88 |
| Total | Control | 6.47 | 6.87 | 7.28 |
| anaerobes | Aleurone | 6.88 | 6.83 | 7.10 |
| Bifidobacteria | Control | 5.99 | 5.55 | 5.39 |
| | Aleurone | 6.13 | 6.37 | 7.03 |
| Lactobacilli | Control | 4.62 | 4.69 | 4.42 |
| | Aleurone | 4.26 | 4.43 | 4.22 |

Short chain fatty acid production by the intestinal microbial community consisted mainly of acetate, propionate, and butyrate with small amounts of isobutyric, valeric, isovaleric, and caproic acid. Supplementation of aleurone in the liquid medium induced specific changes in the fatty acid profiles, mainly in the ascending colon compartment (FIG. 3). Total fatty acid production remained virtually unchanged whereas acetic acid increased, propionate decreased slightly, and butyric acid increased in the ascending colon compartment, but remained virtually unchanged in the transverse and descending colon compartments.

Potential prebiotic properties for humans of aleurone were confirmed using a dynamic in vitro setup of the gastrointestinal tract (SHIME). Endpoints from this example indicate possible prebiotic activities of aleurone, as shown by relative increases in beneficial bacterial species in the intestinal microbial ecosystem and the relative increase of health beneficial bacterial metabolites. Plate count data from the different SHIME compartments indicated bifidobacterial species increased 0.5-, 1-, and 2-logs in the ascending, transverse, and descending colon regions, respectively. Acetate and butyrate increased during the test phase of these experiments indicating aleurone is consumed by a limited number of bacteria, i.e. the acetate-producing bifidobacteria. Butyrate production increased in the ascending colon compartment during the aleurone-feeding phase, but remained steady in the transverse and descending colon. Other bacteria in those regions may have utilized the butyrate as a substrate for growth.

### Example 4

### Effect of Feeding Aleurone in Pet Food on Canine Feces Characteristics

The objective of this experiment was to determine if aleurone was palatable and influenced the feces characteristics of dogs.

### Materials and Methods

Four mixed breed dogs aging from 1 year to 8 years old were used for these experiments. Each dog was fed a prescription dog food containing predominantly duck and rice. The diet was void of any additional fibers. Aleurone was top dressed on the diet at a rate of 0, 1, and 2%. Dogs were fed the National Research Council recommended quantity of food to maintain their body weight. A standard randomized, crossover experimental design was used. Dogs were acclimated to the diet for two weeks before the feeding trials began. Each feeding period lasted 2 weeks with a 2-week washout period between test variables.

Feces quality was subjectively graded on a 7-point scale with one being rock solid and 7 being a consistency of water. A good quality feces (smooth, shiny appearance) was scored with a “3”. Feces were scored on every bowel movement. Feces pH was measured on samples taken at day 0, 7, and 14 in each variable testing period. A completely voided sample was used for each test. Thoroughly mixed feces samples (10 g) were solublized in distilled, deionized water (90 g), and pH was measured using a standardized laboratory pH meter. Data was analyzed by ANOVA.

### Results and Discussion

All four dogs ate 100% of the offered food over the 12-week period. No adverse effects were observed during the feeding trials.

Feces quality improved with both levels of aleurone. The average fecal score for the control variable (0% aleurone) was 2.43; 1% aleurone scored a 3.21, and 2% aleurone scored a 2.68. There was no significant difference in feces quality between individual days (P>0.05).

Feces pH had minor changes when aleurone was fed to the dogs. The feces pH during the run-in periods averaged 5.92. Aleurone at 1 and 2% lowered pH to 5.68 and 5.71, respectively.

Aleurone is well accepted by dogs consuming food top dressed with the fiber. Feces quality improved while consuming the fiber with the 1% inclusion level being more beneficial than the 2% inclusion level. Feces quality went from generally a dry appearance to a shiny, moist appearance. This indicates improved laxation for the dogs. There did not appear to be an adaptation period with time given no statistical difference in feces quality between individual days. An additional indicator of improved bowel health was the lower fecal pH indicating more fermentative species were present in the large intestine.

What is claimed:

1. A method of treating a human or an animal comprising introducing wheat aleurone in the diet of the human or animal.

2. The method of claim 1, wherein the wheat aleurone is introduced in an amount that maintains or improves good feces quality of the human or animal.

3. The method of claim 1, wherein the wheat aleurone is introduced in an amount that maintains or improves good gastrointestinal tract health of the human or animal.

4. The method of claim 1, wherein the human or animal is a mammal and the aleurone is delivered orally.

5. The method of claim 1, wherein the human or animal is a mammal selected from the group consisting of a dog, cat, or horse.

6. The method of claim 1, wherein the human or animal is a mammal and the aleurone is introduced in an amount of about at least 0.1 to 2% per day of the mammal's total daily intake.

7. The method in claim 1, wherein the human or animal is a mammal and the aleurone is introduced in an amount of about 0.5 to 1.5% of the mammal's total daily intake.

8. The method of claim 1, wherein the human or animal is a mammal and the aleurone is introduced in an amount of about 1% of the mammal's total daily intake.

9. A method of improving one or more indicators of bowel health or metabolic health in a companion animal, poultry or fish comprising the step of delivering to the gastrointestinal tract of said animal, poultry or fish an effective amount of aleurone.

10. The method of claim 9, wherein one or more improved indicators of bowel health comprise i) decreased pH of the bowel contents, ii) increased total SCFA concentration or total SCFA amount in the bowel contents, iii) increase in concentration or amount of one or more SCFAs in the bowel contents, iv) increased fecal quality, v) increase in total water volume of bowel or feces, yl) improved laxation, vii) increase in number of activity of one or more species of probiotic bacteria, viii) increase in fecal bile acid excretion, ix) reduced urinary levels of putrefactive products, x) reduced fecal levels of putrefactive products, xi) increased proliferation of normal colonocytes, or xii) any combination of the above.

11. The method of claim 9 wherein one or more indicators is selected from the group consisting of i) decreased pH of the bowel contents, ii) increased total SCFA concentration or total SCFA amount in the bowel contents, iii) increase in concentration or amount of one or more SCFAs in the bowel contents, iv) increased fecal bulk, and v) increase in total water volume of bowel or feces.

12. A food product suitable for humans or animals comprising wheat-derived aleurone in said food product in an amount that provides good feces quality, good gastrointestinal health or a combination thereof.

13. The food product of claim 12 wherein said animals are selected from the group consisting of companion animals, poultry and fish.

14. The food product of claim 12 wherein said animals are mammals.

15. The food product of claim 14 wherein said wheat-derived aleurone is included in said food product in an amount suitable for 0.5% to 1.5% of the mammal's total daily intake.
